(19) United States
(12) Patent Application Publication
(10) Pub. No.: US 2020/0093704 A1
(43) Pub. Date: Mar. 26, 2020

(54) Automatic medication dispenser

(71) Applicant: Alain Kibasosa Ghonda Sr, Silver Spring, MD (US)

(72) Inventor: Alain Kibasosa Ghonda Sr, Silver Spring, MD (US)

(21) Appl. No.: 16/350,078

(22) Filed: Sep. 24, 2018

(51) Int. Cl. A61J 7/04; G16H 20/13

(52) U.S. Cl. CPC A61J 7/049; A61J 2200/30; G16H 20/13; A61J 7/0454

(57) ABSTRACT

An automatic medication dispenser device includes a container structure for containing medications (pills or liquids). There is a display screen on the container displaying the current date and time, name of patient (user), prescription name and dosage, when the prior dose was taken and when the next one is due. A button is push to activate the dispensing of the medication.

The provider can control the AMD remotely: proper use of the medication and interact through video (telemedicine) with the patient. Only a provider such as (doctor, nurses or pharmacist) will be able to access the area where medicines are contained since it requires a special key.

### BACKGROUND OF THE INVENTION

America is going through a serious opioid crisis, where hundreds of thousands are losing their lives and society is spending billions of dollars to try to help them. We also have accidental death, when someone mistakenly takes another person's medications.

### STORY OF THE INVENTION

Different people have tried to bring a solution to this problem without much success this far.

Some examples:
Pill cap timer: it only opens at a certain time; however, the patient can still take more pills than prescribed and abuse it at a later time.
Automatic Pill Dispenser: no security around it, the device dispenses the medication or unlocks itself when it is time for the next dose, anyone can pick up the medication and take it, or simply access the device prior to the patient and abuse the medication.

As a parent of a child with a chronic disease that causes her to take controlled pain medications, this product is to serve my family and the needs of many others.

Today, we bring a solution that can help patients avoid taking more than prescribed medication and also avoid the wrong person taking a medication not prescribe to them.

Our design provides a controlled access to the medication: dispenser is regulated by a timer and fingerprint scanner or passcode. This can only be activated by the proper identification of the patient and when dosage is due. This will be for both pills and liquid medications.

This invention is geared towards patients who are prescribed medications such as controlled substances (opioids and risky others prescriptions), but also for people who need reminder to take their medications (senior citizens, Alzheimer & ADHD patients or just those who are just don't want to risk missing a dose).

### BRIEF DESCRIPTION OF THE DRAWINGS

FIG. 1 is a perspective view of the assembled preferred embodiment of the automatic medication dispenser device according to the present invention.

FIG. 2 is a rendering view of the inside of the pill dispenser

FIG. 3 is a rendering view of the inside of the liquid dispenser

### DETAILED DESCRIPTION OF THE DEVICE

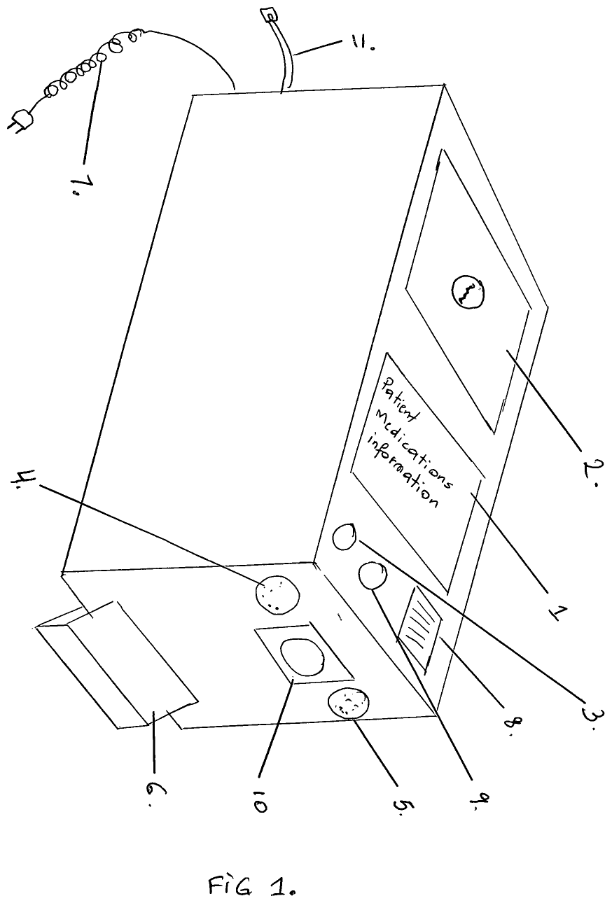

FIG. 1: 1. Digital display screen: displays all required information on a prescription bottle, i.e. name, date filled, expiration date, dosage, quantity, etc.) 2. Door through which providers (doctors or pharmacists) can refill and lock the access 3. Light: changes colors (red all the time and turns green when it is time and ready to dispense) 4. Button to display information about the content and also to activate dispensing of the medication 5. Speaker: vocal reminder or vocal display of the content of the section 6. Receiving tray for pills 7. Power cord (electricity 8. Fingerprint scanner 9. Bluetooth Connection 10. Camera 11. Ethernet connection (plug/cord)

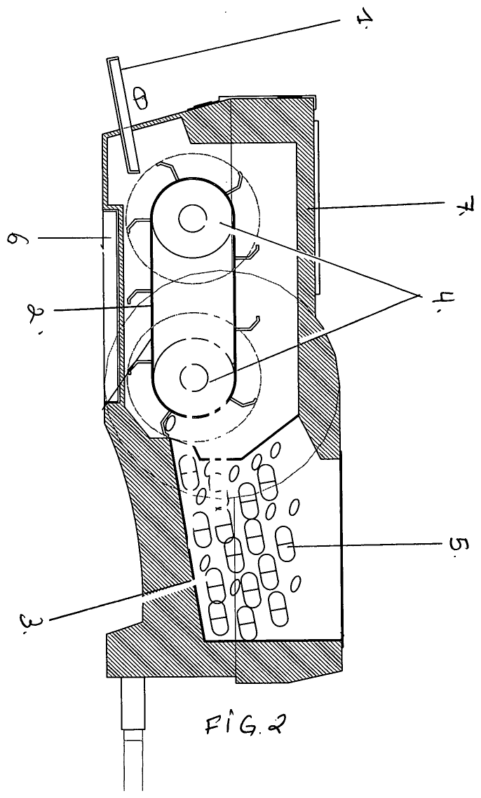

FIG. 2: 1. receiving tray (removable) 2. Conveyor belt: this moves the pills from the medication container and drops it in the receiving tray 3. Medication container: filled by providers only 4. Pulleys: used to move the conveyor belt 5. Sample of pills 6. Built-in batteries (recharges when the device plugged) 7. Electronic compartment

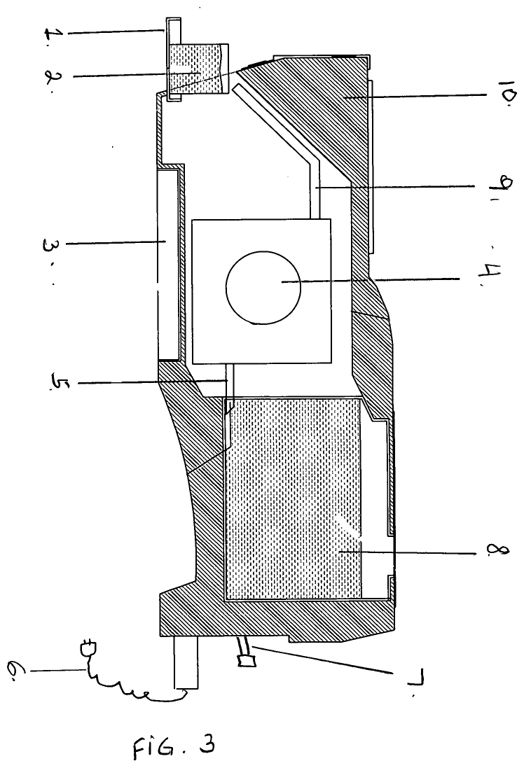

FIG. 3: 1. Receiving tray: where cup is placed 2: Medication cup: receiving the medication 3. Power compartment and battery 4. Pump: inside there is a hydraulic/mechanic pump that takes the liquid from the container and dispenses it 5. Tube/Pipe: connected to the container 6. Power cord: to connect to electrical outlet 7. Ethernet Plug: to connect to the internet 8. Medication Container (for liquid) 9. Tube/pipe: connects to the pump and dispenses the medication 10. Electronic compartment: where all electronic components are stored.

1. Claim 1: The Automatic Medication Dispenser is a smart medication dispenser.

2. The Automatic dispenser of claim 1, where the device is equipped with smart software or Artificial Intelligence (AI) that helps it communicate remotely with the providers (healthcare providers or pharmacists) thru the internet.

3. The Automatic dispenser of claim 1, where the device software keeps track of the medication adherence (intake or missed doses).

4. The Automatic dispenser of claim 1, where the device reminds the user when it is time to take the medication and notifies the listed caregiver when a dose is missed and user failed to act on reminders.

5. The Automatic dispenser of claim 1, where the AMD may have multiple prescriptions in multiple containers; AMD will know which medication to dispense at the specific time and which container to dispense from based on the prescription.

6. Claim 2: Cancer treatment: The Automatic dispenser of claim (the liquid version) of the machine could be used for cancer patients who need to receive intravenous (IV) medications such as: chemotherapy, biological immunotherapy, pain medication or other fluids not requiring them to be in medical facility.

7. The Automatic dispenser of claim 2, where the provider could monitor the patient through the video feed from the built in camera.

8. The Automatic dispenser of claim 2, where the provider can remotely control the flow of the medication (adjust or stop it if necessary).

9. Claim 3: Remote Control and Safeguard: AMD permits for the providers (medical corps or caregiver) to monitor the patient's medication intake by logging in to the platform.

10. The Automatic dispenser of claim 3, where the AMD, the provider can modify or stop the dispensation.

11. The Automatic dispenser of claim 3, where the AMD, thru Artificial Intelligence is also monitoring the flow (liquid version) of the medication, failsafe procedure (shut off the pump) will automatically activate if the machine malfunctions for any reason.

12. Claim 4: Connection with Peripherals: AMD can connect with other peripherals such as: thermometer, glucometer, blood pressure monitor, etc. and record the vitals.

13. The Automatic dispenser of claim 4, where AMD can use its AI to compare with guidelines and make recommendations such (referring to a provider or medication to take.

14. The Automatic dispenser of claim 4, where AMD through the built in camera could be used to monitor the dispensation of the medications and also by the provider for telemedicine.
